(19) United States
(12) Patent Application Publication
(10) Pub. No.: US 2010/0324075 A1
(43) Pub. Date: Dec. 23, 2010

(54) THERAPEUTIC USE OF CARBOXYL ESTER LIPASE INHIBITORS

(75) Inventors: David Y. Hui, Cincinnati, OH (US); Philip N. Howles, Cincinnati, OH (US)

Correspondence Address: DINSMORE & SHOHL LLP, 1900 CHEMED CENTER, 255 EAST FIFTH STREET, CINCINNATI, OH, 45202, US

(73) Assignee: UNIVERSITY OF CINCINNATI, Cincinnati, OH (US)

(21) Appl. No.: 12/809,630

(22) PCT Filed: Dec. 19, 2008
(86) PCT No.: PCT/US08/87669

§ 371 Date: Sep. 13, 2010

Related U.S. Application Data
(60) Provisional application No. 61/015,899, filed on Dec. 21, 2007.
(60) Provisional application No. 61/092,496, filed on Aug. 28, 2008.

(51) Int. Cl. A61K 31/505; A61K 31/27; A61P 9/12; A61P 9/10; A61P 3/06; A61P 1/16; A61K 31/445; A61K 31/40; A61K 31/4418; A61K 31/405; A61K 31/366; A61K 31/47

(52) U.S. Cl. 514/275; 514/490; 514/479; 514/330; 514/423; 514/277; 514/419; 514/460; 514/311

(57) ABSTRACT

Compounds, pharmaceutical compositions, and their methods of use in raising serum HDL levels in subjects in need thereof are provided. Also provided are compounds, pharmaceutical compositions, and their methods of use in increasing reverse cholesterol transport in subjects in need thereof. Finally, methods for treating disease by raising serum HDL levels and increasing reverse cholesterol transport are provided.

This application claims the benefit of U.S. Provisional Application No. 61/015,899, filed Dec. 21, 2007, and U.S. Provisional Application No. 61/092,496 filed Aug. 28, 2008, which applications are hereby incorporated by reference in their entirety.

The present invention relates to methods of raising serum high density lipoprotein (HDL) levels and increasing reverse cholesterol transport comprising administering a safe and effective amount of at least one carboxyl ester lipase (CEL) inhibitor. The invention further relates to pharmaceutical compositions comprising CEL inhibitors and their methods of use in raising serum HDL levels and increasing reverse cholesterol transport. The invention further relates to methods for treating disease by raising serum HDL levels and increasing reverse cholesterol transport.

High density lipoproteins (HDL) comprise one of the three major classes of lipoproteins which transport lipids in the blood stream. The beneficial effects of HDL derive, in part, from their central role in reverse cholesterol transport, which is the movement of cholesterol from peripheral tissues to the liver where it is removed from circulation and eliminated from the body as biliary cholesterol and bile acids. In recent years, considerable attention has been focused on understanding the mechanisms that underlie this overall process, with the goal of developing means to enhance reverse cholesterol transport as a means of treating, preventing, or reducing risk factors for atherosclerosis.

Much of this attention has been focused on the regulation of cholesterol efflux from macrophage foam cells to nascent or circulating HDL particles. Data show that efflux is directly affected by changing expression of the cholesterol transporters ABCG1 and ABCA1. Scavenger receptor-BI (SR-BI) also plays a role.

Altering the amount and type of cholesterol acceptor in the plasma also affects efflux rates and reverse cholesterol transport. Lipid-poor apoAI and pre-β HDL, for example, greatly enhance the process and can promote plaque regression. In recent years, attention has been devoted to methods of raising HDL so as to increase the total vehicle capacity for reverse cholesterol transport, and apoAI mimetics have shown some promise in this regard. Raising HDL levels has also been the focus of cholesteryl ester transfer protein (CETP) inhibitors which prevent the cholesteryl ester (CE): triglyceride exchange between HDL and remnant lipoproteins. However, it has not been established that simply raising HDL cholesterol enhances reverse cholesterol transport. Rather, studies suggest that size and/or composition of the particles significantly affects their ability to promote cholesterol efflux. In addition, both animal and human studies show that raising HDL by CETP inhibition may increase or decrease reverse cholesterol transport, as measured by fecal sterol disposal, depending on genetic and other factors that affect lipoprotein properties and clearance rates.

Other work has focused on hepatic uptake and metabolism of cholesterol and CE from mature HDL. It is now well-accepted that SR-BI plays a major role in the selective uptake of HDL-CE by hepatocytes and that HDL cholesterol is largely targeted for biliary sterols. The importance of SR-BI to the reverse cholesterol transport process has been demonstrated by experiments showing that fecal disposal of macrophage cholesterol is greatly reduced in the absence of this protein. Approximately 75% of HDL cholesterol is esterified and must be hydrolyzed to free cholesterol by the liver before being secreted directly into the bile or being converted to bile salts before secretion. Thus, hydrolysis is believed to be an important first step in the hepatic processing of HDL-CE for the last critical stage of reverse cholesterol transport. The identity of the lipase(s) responsible for this hydrolysis is not clear. One candidate is the enzyme carboxyl ester lipase (CEL), also called bile salt-dependent lipase, bile salt-stimulated lipase, or cholesterol esterase.

CEL is an esterolytic enzyme with wide substrate reactivity capable of hydrolyzing cholesteryl esters, acylglycerols, and lysophospholipids. CEL also possesses lipoamidase activity capable of hydrolyzing ceramides and liberating lipoic acids covalently bound to ε-amino groups of lysine residues in proteins. CEL is synthesized predominantly in pancreatic acinar cells and secreted into the intestinal lumen in response to food intake and early studies implied a role for CEL in mediating the absorption of dietary cholesterol and fat-soluble vitamins in the gastrointestinal tract. However, more recent studies with CEL-deficient mice clearly showed that CEL is only required for the absorption of dietary cholesteryl esters and plays only an auxiliary role in absorption of dietary fat, nonesterified cholesterol, and fat-soluble vitamins. Since cholesteryl esters constitute a very small percentage of total cholesterol in the diet, the impact of CEL deficiency on the total dietary cholesterol absorption is minimal.

CEL is also synthesized in the liver where it can be found intracellularly or secreted into the plasma circulation. Previous work by the inventors with cell culture models and gene knockout mice showed that CEL is associated with the SR-BI pathway in hepatocytes and that it plays a significant role in the hydrolysis of HDL-CE during or immediately after selective uptake via SR-BI (Camarota, et al., Carboxyl ester lipase cofractionates with scavenger receptor BI in hepatocyte lipid rafts and enhances selective uptake and hydrolysis of cholesteryl esters from HDL3. J. Biol. Chem. 279:42889-905 (2004)).

Provided herein are comparative data from Cel^{-/-} and control mice that further demonstrate the role of this enzyme in HDL metabolism. Results show markedly increased reverse cholesterol transport and fecal disposal of HDL- as well as macrophage-derived CE in the absence of CEL due to a combination of increased secretion of unhydrolyzed HDL-CE directly into bile and decreased hydrolysis and reabsorption of this CE by the intestine. Results also show increased plasma HDL cholesterol in Cel^{-/-} mice expressing CETP as well as increased excretion of HDL cholesterol in this animal model.

Also provided herein are data from mice fed an inhibitor of CEL, demonstrating that chemical inhibition of the enzyme increases reverse cholesterol transport to a similar degree as obtained with Cel^{-/-} mice.

Accordingly, one aspect of the present invention indicates that loss of CEL function causes increased reverse cholesterol transport, increased removal of cholesterol from macrophage cells, and elimination of this cholesterol in feces via the liver. These findings are in contrast to suggestions from other teachings which showed reduced selective uptake of HDL-CE by cultured hepatocytes in the absence of CEL. A second aspect of the present invention indicates that loss of CEL function causes increased levels of HDL cholesterol in subjects with suboptimal levels of this lipoprotein.

Together, these results indicate that therapy with a CEL inhibitor raises serum HDL levels and increases reverse cholesterol transport, which may prevent or reverse deposition of cholesterol as arterial plaque and have a beneficial effect on diseases such as atherosclerosis, hyperlipidemia, hypercholesterolemia, cholestatic liver disease, peripheral vascular disease, dyslipidemia, hyperbetalipoproteinemia, hypoalphalipoproteinemia, hypertriglyceridemia, familial-hypercholesterolemia, cardiovascular disorders, angina, ischemia, cardiac schemia, stroke, myocardial infarction, reperfusion injury, angioplastic restenosis, hypertension, vascular complications of diabetes, obesity and endotoxemia. Further, the data indicate that low-dose statin treatment in combination with a CEL inhibitor lowers plasma cholesterol by decreasing cholesterol synthesis and by increasing reverse cholesterol transport, and is thereby an effective treatment for a host of diseases which are beneficially served by these outcomes, including but not limited to, hypercholesterolemia and dyslipidemia.

CEL inhibitors, including CEL-inhibiting compounds and antisense RNA, are useful in raising serum HDL levels and increasing reverse cholesterol transport in subjects in need thereof.

Accordingly, it is an object of the invention to provide a method of raising serum HDL levels comprising administering to a subject in need thereof a safe and effective amount of at least one CEL inhibitor.

Another object of the invention is to provide a pharmaceutical composition comprising at least one CEL inhibitor and at least one pharmaceutically-acceptable carrier, wherein said pharmaceutical composition acts to raise serum HDL levels in a subject in need thereof.

Still another object of the invention is to provide a method of increasing reverse cholesterol transport comprising administering to a subject in need thereof a safe and effective amount of at least one CEL inhibitor.

Another object of the invention is to provide a pharmaceutical composition comprising at least one CEL inhibitor and at least one pharmaceutically-acceptable carrier, wherein said pharmaceutical composition acts to increase reverse cholesterol transport in a subject in need thereof.

Still another object of the invention is to provide a method for treating disease by increasing reverse cholesterol transport, wherein the disease is selected from the group consisting of atherosclerosis, hyperlipidemia, hypercholesterolemia, cholestatic liver disease, peripheral vascular disease, dyslipidemia, hyperbetalipoproteinemia, hypoalphalipoproteinemia, hypertriglyceridemia, familial-hypercholesterolemia, cardiovascular disorders, angina, ischemia, cardiac schemia, stroke, myocardial infarction, reperfusion injury, angioplastic restenosis, hypertension, vascular complications of diabetes, obesity and endotoxemia, which comprises administering a safe and effective amount of a CEL inhibitor to a subject in need thereof.

Another object of the invention is to provide a method for treating disease by raising serum HDL levels, wherein the disease is selected from the group consisting of atherosclerosis, hyperlipidemia, hypercholesterolemia, cholestatic liver disease, peripheral vascular disease, dyslipidemia, hyperbetalipoproteinemia, hypoalphalipoproteinemia, hypertriglyceridemia, familial-hypercholesterolemia, cardiovascular disorders, angina, ischemia, cardiac schemia, stroke, myocardial infarction, reperfusion injury, angioplastic restenosis, hypertension, vascular complications of diabetes, obesity and endotoxemia, which comprises administering a safe and effective amount of a CEL inhibitor to a subject in need thereof.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified.

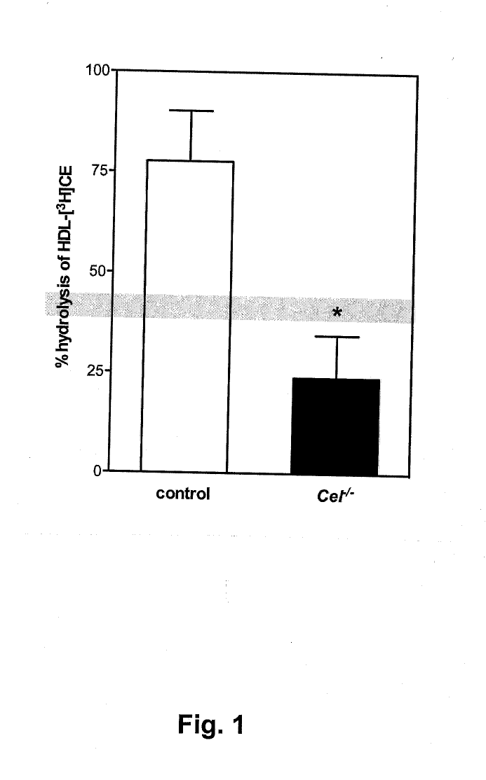

FIG. 1. Hydrolysis of HDL-CE is reduced in hepatic bile from Cel^{-/-} mice. Flowing hepatic bile was collected for 1 h after injection of HDL-[³H]CE into the vena cava of anesthetized mice. Lipids were resolved by TLC to separate [³H]cholesterol from [³H]cholesteryl ester. Data are presented as the percent of radiolabeled biliary cholesterol that was hydrolyzed from. n=4 control and 7 Cel^{-/-} mice. *P<0.0001.

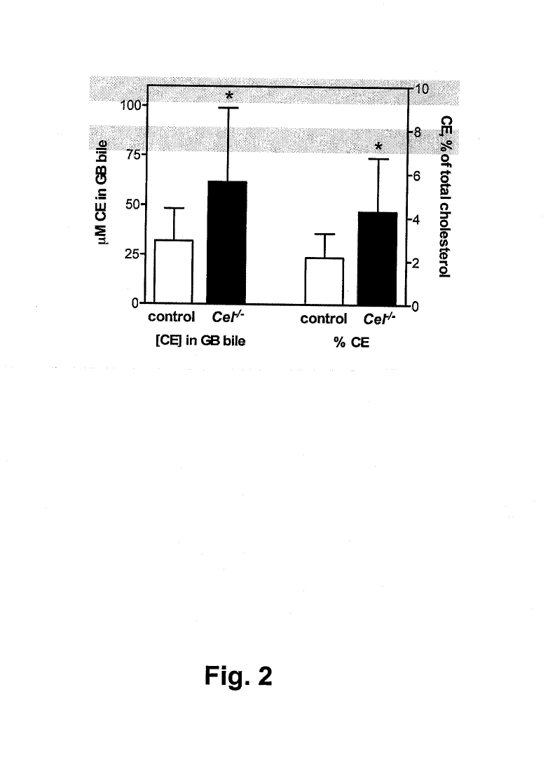

FIG. 2. Cholesteryl ester content of gall bladder bile is greater in Cel^{-/-} mice. Gall bladders were taken midway through the light cycle. Free and esterified cholesterol were determined by direct enzymatic assay. n=8 control and 13 Cel^{-/-} mice. *P<0.02 vs. controls. GB, gall bladder.

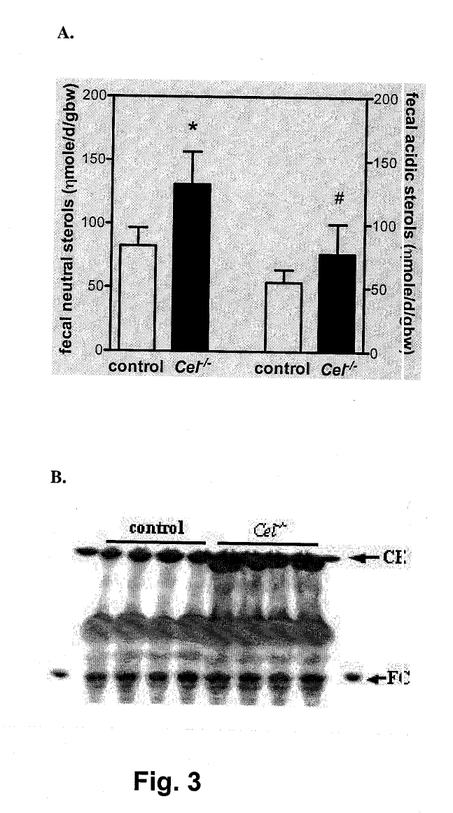

FIG. 3. Excretion of HDL-CE is increased in Cel^{-/-} mice and the sterol remains esterified. Total fecal output was collected for 24 h after tail vein injection of radiolabeled HDL-[³H]CE. Neutral sterols were extracted and radiolabel in free vs. esterified was resolved by TLC and quantitated. n=6 control and 4 Cel^{-/-} mice. *P<0.006 vs. controls.

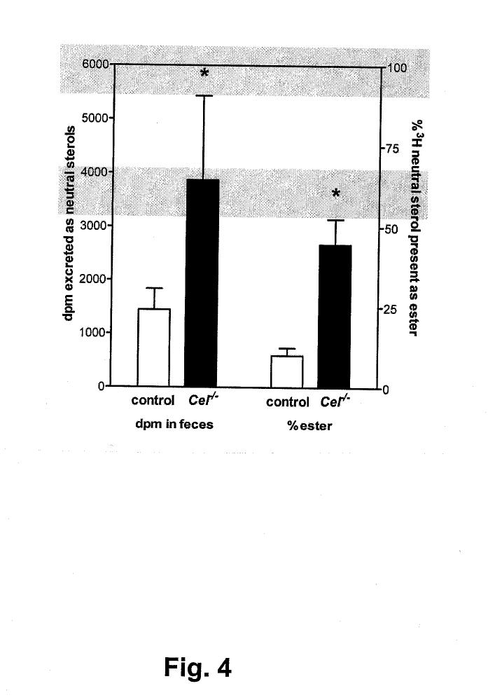

FIG. 4. Excretion of HDL-CE is increased in Cel^{-/-} mice and the sterol remains esterified. Total fecal output was collected for 24 h after tail vein injection of radiolabeled HDL-[³H]CE. Neutral sterols were extracted and radiolabel in free vs. esterified cholesterol was resolved by TLC and quantitated. n=6 control and 4 Cel^{-/-} mice. *P<0.006 vs. controls.

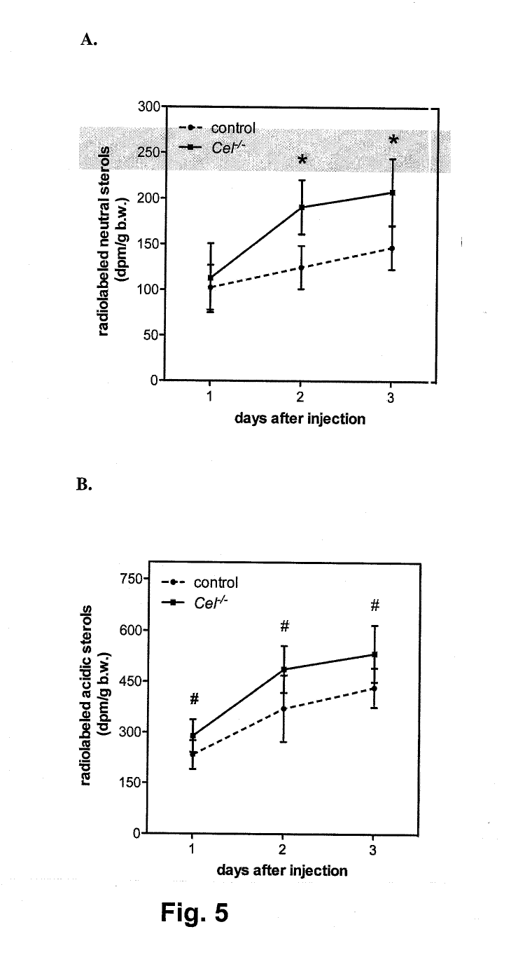

FIG. 5. Reverse cholesterol transport of macrophage cholesterol is greater in Cel^{-/-} mice. Cells of the J774 mouse macrophage line were loaded with acetylated LDL prelabeled with [³H]cholesteryl oleate and given to mice by intraperitoneal injection. Total fecal output was collected for the subsequent 3 days and radiolabel in neutral (A) and acidic (B) sterol was quantitated. n=8 control and 10 Cel^{-/-} mice. *P<0.001, ^{#}P≦0.023.

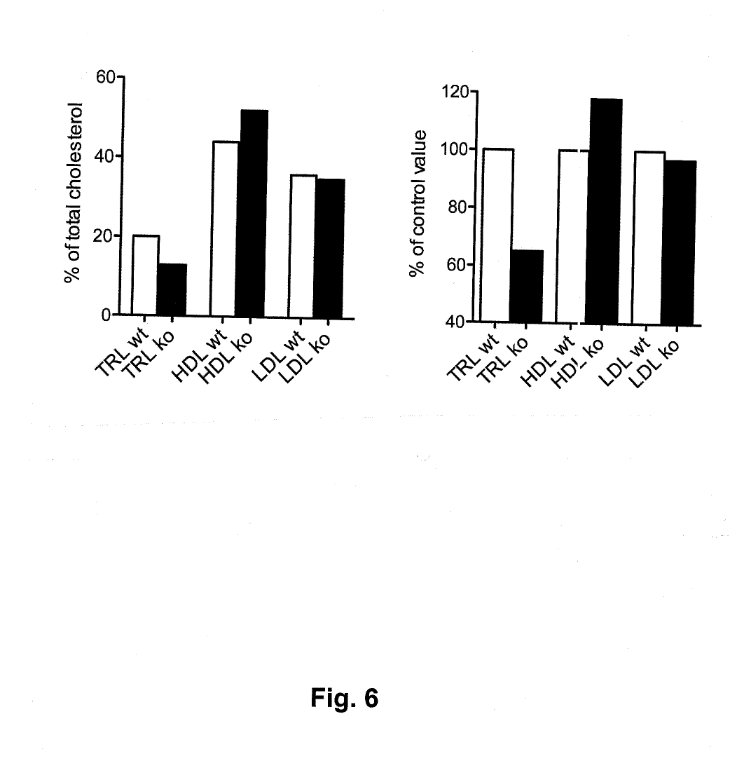

FIG. 6. Atheroprotective HDL cholesterol is increased and atherogenic VLDL cholesterol is decreased in Cel^{-/-} mice expressing CETP. Pooled plasma from n=10 CETP/control and 15 CETP/Cel^{-/-} mice was fractionated by FPLC and the amount of total cholesterol in each fraction was determined. The relative distribution of cholesterol among the major lipoprotein classes is presented as a fraction of total plasma cholesterol in the left panel. The right panel presents the CETP/Cel^{-/-} data as a percent of CETP/controls to illustrate the change in distribution sue to lack of CEL. TRL, triglyceride rich lipoproteins (VLDL+IDL).

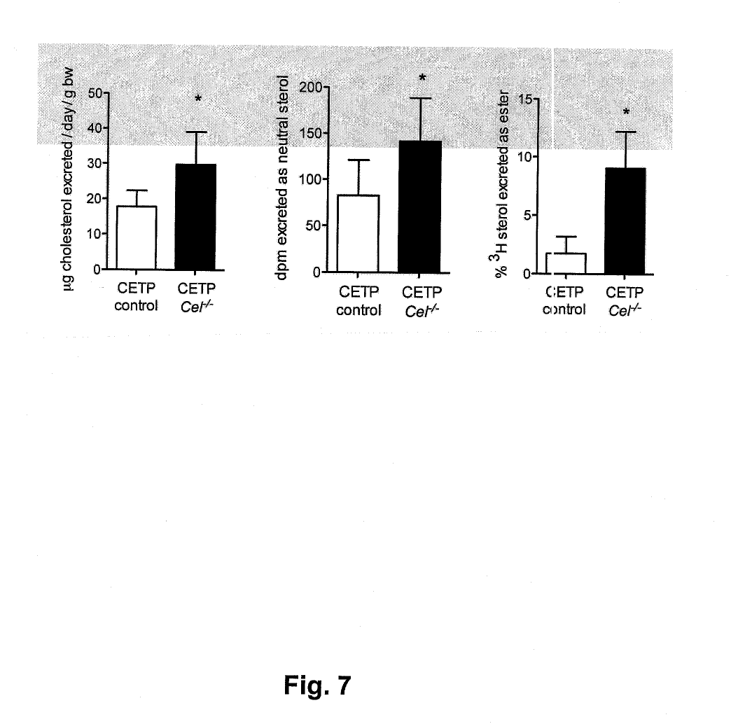

FIG. 7. Excretion of HDL-derived cholesterol as well as total cholesterol mass is increased in Cel^{-/-} mice expressing CETP. Total fecal output was collected for 24 h after retro-orbital injection of radiolabeled HDL-[³H]CE. Total neutral sterols were extracted and quantitated by gas chromatography (left panel). The amount of excreted radiolabel was determined (center panel) and the portion in free vs. esterified cholesterol was resolved by TLC and quantitated (right panel). n=8 CETP/control and 14 CETP/Cel^{-/-} mice. *P<0.008 vs. CETP/controls.

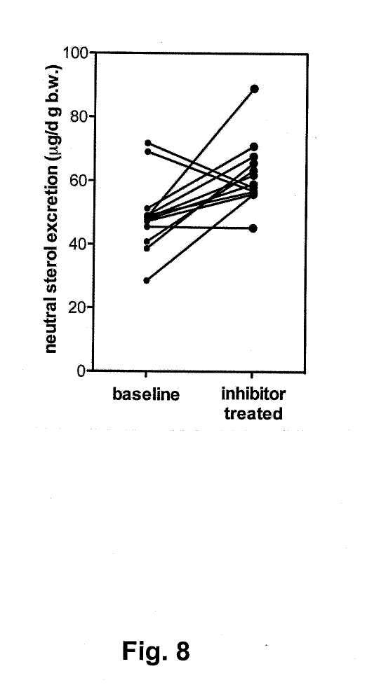

FIG. 8. Excretion of neutral sterol mass is increased by feeding CEL inhibitor (1,5-dimethylhexyl)carbamic acid 4-phenoxyphenyl ester to normal C57BL/6 mice. Inhibitor was mixed with standard rodent chow and fed to mice after baseline excretion was determined. Sterol excretion was again determined after 2 weeks of inhibitor treatment. The change from baseline is plotted for each mouse (n=13).

The following is a list of definitions for terms used herein.

The term “CEL inhibitor,” as used herein, means an agent capable of inhibiting the activity and/or the expression level of the enzyme carboxyl ester lipase. In one aspect of the invention, CEL inhibitors comprise chemical compounds or analogs capable of inhibiting CEL function (“CEL-inhibiting compounds”). In another aspect of the invention, CEL inhibitors comprise antisense RNA capable of inhibiting CEL expression by interfering with the translation of the CEL mRNA into functional CEL protein.

The term “antisense RNA,” as used herein, means modified and unmodified DNA and RNA oligonucleotides, ribozymes, catalytic deoxyribozymes, small interfering RNA (siRNA), short hairpin RNA (shRNA), and the like, which directly target the CEL mRNA for degradation or inhibit its translation.

For the purposes of the present invention, the terms “standard diet,” “standard chow diet,” “basal diet,” and “normal diet” are used interchangeably and mean rodent chow comprising about 4.5% fat, which may be derived from many acceptable sources, including soybean oil.

For the purposes of the present invention the terms “compound” and “analog” stand equally well for the compositions of matter described herein, including all enantiomeric forms, diastereomeric forms, salts, prodrugs, and the like, and the terms “compound” and “analog” are used interchangeably throughout the present specification.

The term “halo” means chloro, bromo, iodo or fluoro. In one embodiment, perhaloalkyl substituents are perfluoroalkyl and the alkyl and alkoxy substituents have from 1 to 4 carbon atoms.

A “pharmaceutically-acceptable salt” is a cationic salt formed at any acidic (e.g., hydroxamic or carboxylic acid) group, or an anionic salt formed at any basic (e.g., amino) group. Many such salts are known in the art, as described in WO 87/05297, by Johnston et al., published Sep. 11, 1987. Specific cationic salts include the alkali metal salts (such as sodium and potassium), and alkaline earth metal salts (such as magnesium and calcium) and organic salts. Specific anionic salts include the halides (such as chloride salts), sulfonates, carboxylates, phosphates, and the like.

Such salts are well understood by the skilled artisan, and the skilled artisan is able to prepare any number of salts given the knowledge in the art. Furthermore, it is recognized that the skilled artisan may select one salt over another for reasons of solubility, stability, formulation ease and the like. Determination and optimization of such salts is within the purview of the skilled artisan's practice.

The terms “enantiomer” and “diastereomer” have the standard art recognized meanings (see, e.g., Hawley's Condensed Chemical Dictionary, 14th ed.). The illustration of specific protected forms and other derivatives of the compounds of the instant invention is not intended to be limiting. The application of other useful protecting groups, salt forms, etc. is within the ability of the skilled artisan.

The term “pharmaceutically-acceptable carrier,” as used herein, means any physiologically inert, pharmacologically inactive material known to one skilled in the art, which is compatible with the physical and chemical characteristics of the particular CEL inhibitor selected for use. Pharmaceutically-acceptable carriers include, but are not limited to, polymers, resins, plasticizers, fillers, lubricants, diluents, binders, disintegrants, solvents, co-solvents, buffer systems, surfactants, preservatives, sweetening agents, flavoring agents, pharmaceutical grade dyes or pigments, and viscosity agents.

The term “safe and effective amount,” as used herein, means an amount of a compound or composition high enough to significantly positively modify the symptoms and/or condition to be treated, but low enough to avoid serious side effects (at a reasonable risk/benefit ratio), within the scope of sound medical judgment. The safe and effective amount of active ingredient for use in the method of the invention herein will vary with the particular condition being treated, the age and physical condition of the patient to be treated, the severity of the condition, the duration of the treatment, the nature of concurrent therapy, the particular active ingredient being employed, the particular pharmaceutically-acceptable carriers utilized, and like factors within the knowledge and expertise of the attending physician.

The term “cholesterol lowering agent,” as used herein, means any dietary supplement, compound, analog, drug, prodrug, enantiomer, diastereomer, or salt thereof which can be administered to a subject in order to reduce serum cholesterol levels. Cholesterol lowering agents include, but are not limited to, statin drugs; drugs which block cholesterol absorption, such as ezetimibe and related compounds; bile acid sequestrants, such as colesevelam, cholestyramine, colestipol, and niacin (nicotinic acid); and fibric acid derivatives such as gemfibrozil, fenofibrate, and clofibrate.

The terms “statin” and “statin drug,” as used herein, refer to the class of HMG-CoA reductase inhibitors which are useful in lowering cholesterol levels in individuals who have or are at risk for cardiovascular diseases such as atherosclerosis, hypercholesterolemia, hyperlipidemia, and the like. Statin drugs include, but are not limited to, atorvastatin, cerivastatin, fluvastatin, lovastatin, mevastatin, pitavastatin, pravastatin, rosuvastatin, and simvastatin.

The term “subject,” as used herein, means any mammalian subject, including humans.

The terms “treat,” “treatment,” and “treating,” as used herein, refer to a method of alleviating or abrogating a disease, disorder, and/or symptoms thereof.

The terms “prevent,” “prevention,” and “preventing,” as used herein, refer to a method of barring a subject from acquiring a disease, disorder, and/or symptoms thereof. In certain embodiments, “prevent,” “prevention,” and “preventing” refer to a method of reducing the risk of acquiring a disease, disorder, and/or symptoms thereof.

The term “reverse cholesterol transport,” as used herein, refers to the process by which cholesterol is removed from tissues other than the liver and is carried back to the liver for disposal via the bile as biliary cholesterol or as bile salts.

The term “HDL,” as used herein, refers to high density lipoproteins, which enable lipids such as cholesterol and triglycerides to be transported in the water based blood stream. This includes the subclassifications known as HDL₂ and HDL₃ and preβ-HDL. HDL-bound cholesterol, or HDL-C, is sometimes referred to as “good” cholesterol, since HDL is believed to play a role in transporting cholesterol back to the liver for excretion or re-use.

### Therapeutic CEL-Inhibiting Compounds

Surprisingly, various known compounds have been found to exhibit CEL inhibitory properties. See, for example, the compounds disclosed in U.S. Pat. No. 5,391,571, issued Feb. 21, 1995 to Mewshaw et al.; U.S. Pat. No. 5,512,565, issued Apr. 30, 1996 to Mewshaw et al.; U.S. Pat. No. 5,942,631, issued Aug. 24, 1999, to Deck et al.; U.S. Pat. No. 6,034,255, issued Mar. 7, 2000, to Deck et al.; U.S. Pat. No. 6,114,545, issued Sep. 5, 2000, to Deck et al.; U.S. Pat. No. 5,017,565, issued May 21, 1991, to Lange, III et al.; and U.S. Pat. No. 5,063,210, issued Nov. 5, 1991, to Lange, III et al. These documents are incorporated herein by reference in their entirety.

In one embodiment of the present invention, compounds include all enantiomeric and diastereomeric forms and pharmaceutically acceptable salts thereof having the Formula:

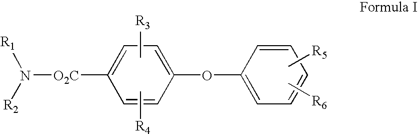

wherein

R₁ is branched or straight chain, saturated or unsaturated alkyl of 4 to 20 carbon atoms, cycloalkyl of 3 to 8 carbon atoms, 1-adamantyl, 2-adamantyl, 3-noradamantyl, 3-methyl-1-adamantyl, 1-fluorenyl, 9-fluorenyl, cycloalkylalkyl where the cycloalkyl moiety has 3 to 8 carbon atoms and the alkyl moiety has 1 to 6 carbon atoms, phenyl, substituted phenyl where the substituents are alkyl of 1 to 6 carbon atoms, alkoxy of 1 to 6 carbon atoms, halo, nitro, cyano or trifluoromethyl, phenylalkyl of 7 to 26 carbon atoms or substituted phenylalkyl, where the alkyl moiety is 1 to 20 carbon atoms and the substituent on the benzene ring is alkyl of 1 to 6 carbon atoms, alkoxy of 1 to 6 carbon atoms, halo, nitro, cyano, trifluoromethyl or phenyl;

R₂ is hydrogen, alkyl of 1 to 6 carbon atoms or R¹ taken with R² and the nitrogen atom to which they are attached form a heterocyclic moiety of the formula:

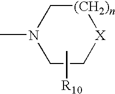

wherein X is

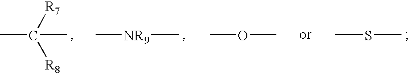

in which

R₇ is hydrogen, branched or straight chain alkyl of 1 to 6 carbon atoms, hydroxy, alkanoyloxy of 2 to 6 carbon atoms, hydroxyalkyl of 1 to 6 carbon atoms, hydroxycarbonyl, alkoxycarbonyl of 1 to 6 carbon atoms, phenyl or substituted phenyl in which the substituents is alkyl of 1 to 6 carbon atoms, alkoxy of 1 to 6 carbon atoms, halo, nitro, cyano, haloalkyl of 1 to 6 carbon atoms, perhaloalkyl of 1 to 6 carbon atoms or dialkylaminoalkyl in which each alkyl group contains from 1 to 6 carbon atoms;

R₈ is hydrogen or branched or straight chain alkyl of 1 to 6 carbon atoms or R₇ and R₈ taken together are polymethylene of 2 to 6 carbon atoms;

R₉ is hydrogen, alkyl of 1 to 6 carbon atoms, phenyl or substituted phenyl in which the substituents is alkyl of 1 to 6 carbon atoms, alkoxy of 1 to 6 carbon atoms, halo, nitro, cyano or perhaloalkyl of 1 to 6 carbon atoms;

R₁₀ is hydrogen, alkyl of 1 to 6 carbon atoms or gemdialkyl of 2 to 12 carbon atoms;

n is one of the integers 0, 1 or 2; and

R₃, R₄, R₅, and R₆ are, independently, hydrogen, branched or straight chain alkyl of 1 to 6 carbon atoms, alkoxy of 1 to 6 carbon atoms, halo, nitro, cyano, perhaloalkyl of 1 to 6 carbon atoms, alkoxycarbonyl of 2 to 16 carbon atoms or hydroxycarbonyl.

In another embodiment of the present invention, the compound is (1,5-dimethylhexyl)carbamic acid 4-phenoxyphenyl ester, having the structure:

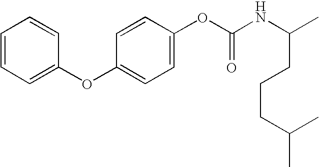

### (1,5-dimethylhexyl)carbamic acid 4-phenoxyphenyl ester

In another embodiment of the invention, the compound is 4-methyl-1-piperidinecarboxylic acid 4-phenoxyphenyl ester, having the structure:

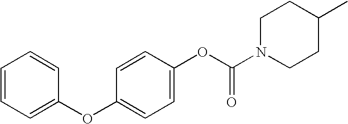

### 4-methyl-1-piperidinecarboxylic acid 4-phenoxyphenyl ester

In still another embodiment of the present invention, compounds include all enantiomeric and diastereomeric forms and pharmaceutically acceptable salts thereof having the Formula II:

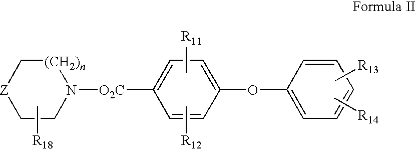

wherein Z is

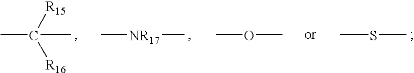

in which

R₁₅ is hydrogen, alkyl, hydroxy, alkanoyloxy, hydroxyalkyl, hydroxycarbonyl, alkoxycarbonyl, phenyl or substituted phenyl, in which the substituent is alkyl, alkoxy, halo, nitro, cyano, haloalkyl, perhaloalkyl or dialkylaminoalkyl;

R₁₆ is hydrogen or alkyl or R₁₅ and R₁₆ taken together are polymethylene;

R₁₇ is hydrogen, alkyl, phenyl or substituted phenyl, in which the substituent is alkyl, alkoxy, halo, nitro, cyano or perhaloalkyl;

R₁₈ is hydrogen, alkyl, or gemdialkyl;

n is one of the integers 0, 1 or 2; and

R₁₁, R₁₂, R₁₃, and R₁₄ are, independently, hydrogen, alkyl, alkoxy, halo, nitro, cyano or perhaloalkyl, alkoxycarbonyl or hydroxycarbonyl.

In another embodiment of the invention, compounds include all enantiomeric and diastereomeric forms and pharmaceutically acceptable salts thereof having the Formula III:

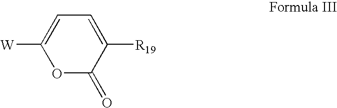

wherein

W is Cl, Br, or I; and

R₁₉ is a member of the group consisting of:

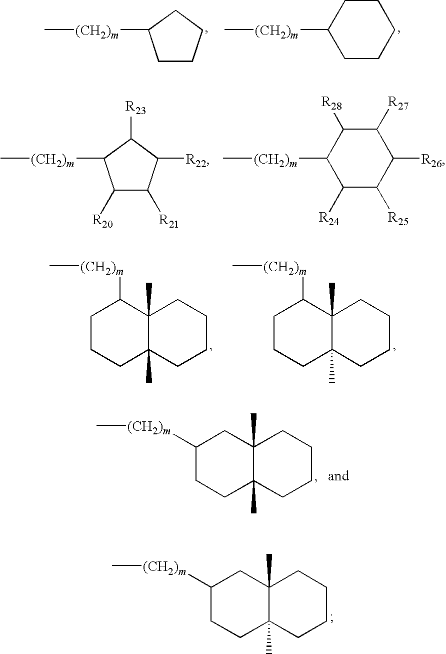

wherein m is one of the integers 0, 1, 2, 3, 4, 5, 6, 7, or 8; and

R₂₀, R₂₁, R₂₂, R₂₃, R₂₄, R₂₅, R₂₆, R₂₇, and R₂₈ are each hydrogen, C₁₋₈ alkyl, C₃₋₈ cycloalkyl, C₂₋₈ alkenyl, or C₂₋₈ alkynyl.

In still another embodiment of the invention, compounds include all enantiomeric and diastereomeric forms and pharmaceutically acceptable salts thereof having the Formula IV:

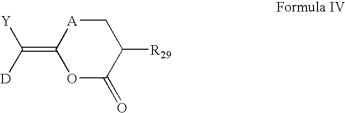

wherein

A is —(CH₂)— where p is 0 or 1;

Y is hydrogen or C₁₋₈ alkyl when D is Cl, Br, or I and Y is Cl, Br, or I when D is hydrogen or C₁₋₈ alkyl; and

R₂₉ is a member of the group consisting of:

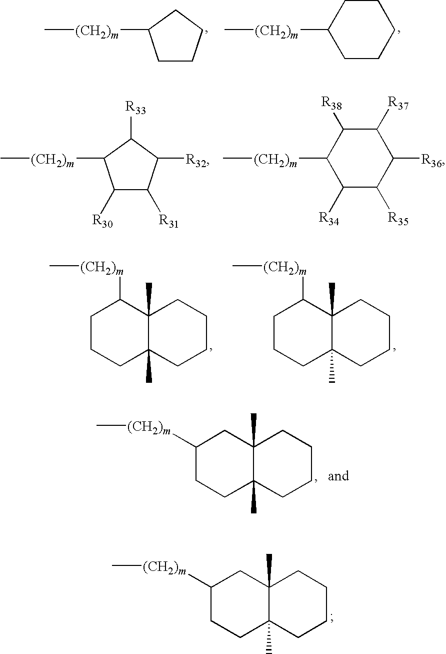

wherein m is one of the integers 0, 1, 2, 3, 4, 5, 6, 7, or 8; and

R₃₀, R₃₁, R₃₂, R₃₃, R₃₄, R₃₅, R₃₆, R₃₇, and R₃₈ are each hydrogen, C₁₋₈ cycloalkyl, C₂₋₈ alkenyl, or C₂₋₈ alkynyl.

In another embodiment of the invention, compounds include all enantiomeric and diastereomeric forms and pharmaceutically acceptable salts thereof having the Formula V:

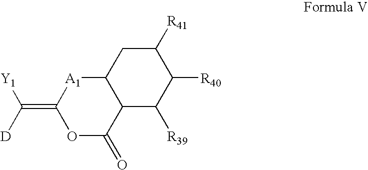

wherein

A₁ is —(CH₂)_{q}— where q is 0 or 1;

Y₁ is hydrogen or C₁₋₈ alkyl when D₁ is Cl, Br, or I and Y₁ is Cl, Br, or I when D₁ is hydrogen or C₁₋₈ alkyl; and

R₃₉, R₄₀, and R₄₁ are each hydrogen, C₁₋₈ cycloalkyl, or C₂₋₈ alkynyl.

Methods of making the compounds described herein above are well known in the art. See, for example, U.S. Pat. Nos. 5,391,571, 5,512,565, 5,942,631, 6,034,255, and 6,114,545.

In still another embodiment of the present invention, compounds include sulfated polysaccharide polymers, such as those described in U.S. Pat. No. 5,017,565, issued May 21, 1991, to Lange, III et al. In a specific embodiment of the invention, the compound is selected from the group consisting of sulfated alginic acid, pectin, amylopectin, chitin, dextran, cellulose agar, and chitosan. In another specific embodiment of the invention, the compound is cellulose sulfate sodium salt, also known as CVT-1 (CAS Registration Number 9005-22-5).

### Antisense RNA as CEL Inhibitors

In one aspect of the present invention, CEL inhibition is accomplished using antisense technology to reduce CEL level by suppressing its expression via targeting of its mRNA. The CEL-specific antisense reagents include unmodified or modified (e.g., by morpholinos, methylation, allylation, phosphorothioate- or phosphoramidite-modified, etc.) short (about 20 nucleotide) DNA or RNA sequence that are complementary to a portion of the CEL mRNA, ribozymes or deoxyribozymes with a catalytic domain flanked by sequences complementary to the CEL mRNA, short double-stranded small interfering RNA with sequence corresponding to a portion of the CEL mRNA, and short hairpin (sh) RNA that are in vivo-processed into functional CEL-specific siRNA. All CEL-specific antisense RNAs are developed based on the CEL mRNA sequence and validated by suppression of CEL expression in CEL-expressing mammalian cells in vitro. The antisense RNA can be delivered as modified or unmodified oligonucleotides, either in naked form, encapsulated in lipid complexes, or attached to fusogenic peptides, antibodies, or cell surface receptor ligands for targeting to specific tissues. Viral vectors including but not limited to adenovirus, adeno-associated virus, retrovirus, and lentivirus may also be used as vehicles for delivery of the antisense RNA, particularly ribozymes, deoxyribozymes, and shRNA, to tissues where CEL is expressed. Route of administration for all of the CEL antisense reagents may be accomplished by intravenous injection. The dose and frequency of CEL antisense reagents necessary to suppress CEL expression can be determined based on the reduction of CEL activity in the blood circulation by ≧50%. Determination of the appropriate dose and mechanism of administration of antisense RNA is within the purview of the skilled artisan.

### Compositions and Dosage Forms

The compositions of this invention are preferably provided in unit dosage form. As used herein, a “unit dosage form” is a composition of this invention containing an amount of a CEL inhibitor that is suitable for administration to subject, more specifically a human subject, in a single dose, according to good medical practice. These compositions preferably contain from about 1 mg to about 750 mg, more preferably from about 3 mg to about 500 mg, still more preferably from about 5 mg to about 300 mg, of a CEL inhibitor.

The compositions of this invention may be in any of a variety of forms, suitable (for example) for oral, rectal, topical, nasal, ocular, transdermal, pulmonary or parenteral administration. Depending upon the particular route of administration desired, a variety of pharmaceutically-acceptable carriers well-known in the art may be used. These include solid or liquid fillers, diluents, hydrotropes, surface-active agents, and encapsulating substances. Optional pharmaceutically-active materials may be included, which do not substantially interfere with the inhibitory activity of the CEL inhibitor. The amount of carrier employed in conjunction with the CEL inhibitor is sufficient to provide a practical quantity of material for administration per unit dose of the compound. Techniques and compositions for making dosage forms useful in the methods of this invention are described in the following references, all incorporated by reference herein: Modern Pharmaceutics, Chapters 9 and 10 (Banker & Rhodes, editors, 1979); Lieberman et al., Pharmaceutical Dosage Forms: Tablets (1981); and Ansel, Introduction to Pharmaceutical Dosage Forms 2d Edition (1976).

Various oral dosage forms can be used, including such solid forms as tablets, capsules, granules and bulk powders. These oral forms comprise a safe and effective amount, usually at least about 5%, and preferably from about 25% to about 50%, of the Formula (I) compound. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin, polyvinylpyrrolidone and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of the subject invention, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, Avicel® RC-591, tragacanth and sodium alginate; typical wetting agents include lecithin and polysorbate 80; and typical preservatives include methyl paraben, propyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject CEL inhibitor is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit® coatings, waxes and shellac.

Compositions of the subject invention may optionally include other active ingredients. Specifically, the additional active ingredient may be a cholesterol lowering agent. Cholesterol lowering agents include any dietary supplement, compound, analog, drug, prodrug, enantiomer, diastereomer, or salt thereof which can be administered to a subject in order to reduce serum cholesterol levels. Cholesterol lowering agents include, but are not limited to, statin drugs, such as atorvastatin, cerivastatin, fluvastatin, lovastatin, mevastatin, pitavastatin, pravastatin, rosuvastatin, and simvastatin; drugs which block cholesterol absorption, such as ezetimibe and related compounds; bile acid sequestrants, such as colesevelam, cholestyramine, colestipol, and niacin (nicotinic acid); and fibric acid derivatives such as gemfibrozil, fenofibrate, and clofibrate.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

In one embodiment of the present invention, a method of raising serum HDL levels is provided, the method comprising administering to a subject in need thereof a safe and effective amount of at least one carboxyl ester lipase (CEL) inhibitor. In another embodiment of the invention, the at least one CEL inhibitor is selected from the group consisting of (1,5-dimethylhexyl)carbamic acid 4-phenoxyphenyl ester and 4-methyl-1-piperidinecarboxylic acid 4-phenoxyphenyl ester.

In another embodiment of the invention, a second active ingredient is co-administered to the subject, wherein the second active ingredient is a cholesterol lowering agent. In one embodiment, the second active ingredient is a statin drug. In another embodiment, the statin drug is selected from the group consisting of atorvastatin, cerivastatin, fluvastatin, lovastatin, mevastatin, pitavastatin, pravastatin, rosuvastatin, and simvastatin.

In another embodiment of the present invention a pharmaceutical composition is provided, the composition comprising:

(a) at least one CEL inhibitor; and

(b) at least one pharmaceutically-acceptable carrier,

wherein said pharmaceutical composition acts to raise serum HDL levels in a subject in need thereof. In one embodiment, the at least one CEL inhibitor is selected from the group consisting of (1,5-dimethylhexyl)carbamic acid 4-phenoxyphenyl ester and 4-methyl-1-piperidinecarboxylic acid 4-phenoxyphenyl ester.

In another embodiment of the invention, the pharmaceutical composition comprises a second active ingredient, wherein the second active ingredient is a cholesterol lowering agent. In one embodiment, the second active ingredient is a statin drug. In another embodiment, the statin drug is selected from the group consisting of atorvastatin, cerivastatin, fluvastatin, lovastatin, mevastatin, pitavastatin, pravastatin, rosuvastatin, and simvastatin.

In another embodiment of the invention, a method of increasing reverse cholesterol transport is provided, the method comprising administering to a subject in need thereof a safe and effective amount of at least one CEL inhibitor. In one embodiment, the at least one CEL inhibitor is selected from the group consisting of (1,5-dimethylhexyl)carbamic acid 4-phenoxyphenyl ester and 4-methyl-1-piperidinecarboxylic acid 4-phenoxyphenyl ester.

In another embodiment, a second active ingredient is co-administered to the subject, wherein the second active ingredient is a cholesterol lowering agent.

In one embodiment, the second active ingredient is a statin drug. In another embodiment, the statin drug is selected from the group consisting of atorvastatin, cerivastatin, fluvastatin, lovastatin, mevastatin, pitavastatin, pravastatin, rosuvastatin, and simvastatin.

In another embodiment of the invention, pharmaceutical composition is provided, the composition comprising:

(a) at least one CEL inhibitor; and

(b) at least one pharmaceutically-acceptable carrier,

wherein said pharmaceutical composition acts to increase reverse cholesterol transport in a subject in need thereof. In one embodiment, the at least one CEL inhibitor is selected from the group consisting of (1,5-dimethylhexyl)carbamic acid 4-phenoxyphenyl ester and 4-methyl-1-piperidinecarboxylic acid 4-phenoxyphenyl ester.

In a further embodiment, the pharmaceutical composition comprises a second active ingredient, wherein the second active ingredient is a cholesterol lowering agent. In another embodiment, the second active ingredient is a statin drug. In still another embodiment, the statin drug is selected from the group consisting of atorvastatin, cerivastatin, fluvastatin, lovastatin, mevastatin, pitavastatin, pravastatin, rosuvastatin, and simvastatin.

In another embodiment of the present invention, a method of increasing reverse cholesterol transport is provided, the method comprising administering to a subject in need thereof a safe and effective amount of a CEL inhibitor selected from the group consisting of (1,5-dimethylhexyl)carbamic acid 4-phenoxyphenyl ester and 4-methyl-1-piperidinecarboxylic acid 4-phenoxyphenyl ester. In another embodiment, a statin drug is co-administered to the subject. In still another embodiment, the statin drug is selected from the group consisting of atorvastatin, cerivastatin, fluvastatin, lovastatin, mevastatin, pitavastatin, pravastatin, rosuvastatin, and simvastatin.

In another embodiment of the invention, a method for treating disease by increasing reverse cholesterol transport, wherein the disease is selected from the group consisting of atherosclerosis, hyperlipidemia, hypercholesterolemia, cholestatic liver disease, peripheral vascular disease, dyslipidemia, hyperbetalipoproteinemia, hypoalphalipoproteinemia, hypertriglyceridemia, familial-hypercholesterolemia, cardiovascular disorders, angina, ischemia, cardiac schemia, stroke, myocardial infarction, reperfusion injury, angioplastic restenosis, hypertension, vascular complications of diabetes, obesity and endotoxemia is provided, the method comprising administering a safe and effective amount of a CEL inhibitor to a subject in need thereof. In another embodiment, the CEL inhibitor is selected from the group consisting of (1,5-dimethylhexyl)carbamic acid 4-phenoxyphenyl ester and 4-methyl-1-piperidinecarboxylic acid 4-phenoxyphenyl ester. In another embodiment, a statin drug is co-administered to the subject. The statin drug may be selected from the group consisting of atorvastatin, cerivastatin, fluvastatin, lovastatin, mevastatin, pitavastatin, pravastatin, rosuvastatin, and simvastatin.

In another embodiment of the invention, a method for treating disease by raising serum HDL levels, wherein the disease is selected from the group consisting of atherosclerosis, hyperlipidemia, hypercholesterolemia, cholestatic liver disease, peripheral vascular disease, dyslipidemia, hyperbetalipoproteinemia, hypoalphalipoproteinemia, hypertriglyceridemia, familial-hypercholesterolemia, cardiovascular disorders, angina, ischemia, cardiac schemia, stroke, myocardial infarction, reperfusion injury, angioplastic restenosis, hypertension, vascular complications of diabetes, obesity and endotoxemia is provided, the method comprising administering a safe and effective amount of a CEL inhibitor to a subject in need thereof. In another embodiment, the CEL inhibitor is selected from the group consisting of (1,5-dimethylhexyl)carbamic acid 4-phenoxyphenyl ester and 4-methyl-1-piperidinecarboxylic acid 4-phenoxyphenyl ester. In another embodiment, a statin drug is co-administered to the subject. The statin drug may be selected from the group consisting of atorvastatin, cerivastatin, fluvastatin, lovastatin, mevastatin, pitavastatin, pravastatin, rosuvastatin, and simvastatin.

### EXAMPLES

The following examples are given by way of illustration, and are in no way intended to limit the scope of the present invention. Data analyses were performed using Prism 5.0 (GraphPad Software, Inc., San Diego, Calif.) and Microsoft Excel 2004. All data are presented as mean±standard deviation. Significance was assigned to differences between groups when t tests yielded P≦0.05.

### Example 1

### Animals and Diets

Control and Cel^{-/-} mice were derived by mating heterozygous males and females. Genotypes were determined by PCR by methods known in the art. The CEL mutation is carried on the C57BL/6J genetic background and is backcrossed to the reference colony (Jackson Laboratories, Bar Harbor, Me.) yearly to minimize genetic drift. Mice were housed in the institution's vivarium with a 12 hr light/dark cycle, controlled temperature and humidity, and ad libitum access to food (Teklad LM485, HarlanTeklad, Madison, Wis.) and water. All procedures were approved by the Institutional Animal Care and Use Committee. All data are derived from experiments performed on male mice.

### Example 2

### Bile Collection and Biliary Clearance of Radiolabeled HDL

Flowing bile was collected from anesthetized mice via a polyethylene catheter inserted into the fundus of the gall bladder as previously described (Huggins, et al., Pancreatic triglyceride lipase deficiency minimally affects dietary fat absorption but dramatically decreases dietary cholesterol absorption in mice. J. Biol. Chem. 278:42899-905 (2003)). Collections were performed approximately midway through the light cycle (11 AM-2 PM) on unfasted animals. Human HDL₃ (1.125<ρ<1.21) was isolated and radiolabeled with [³H]cholesteryl oleate. Up to 750,000 dpm (maximum HDL dose of 0.45 mg protein) was injected into the inferior vena cava just before insertion of the catheter. Bile was collected for 1 hr, and the radiolabel content was determined by scintillation spectrometry. Free and esterified cholesterol were resolved by TLC to determine the relative amount of radiolabel in each fraction.

As shown in FIG. 1, ˜75% (77.6±12.6) of the cholesterol from HDL-[³H]CE was hydrolyzed before secretion into bile, as has been reported previously. However, the opposite was true for Cel^{-/-} mice, in which ˜75% of the cholesterol remained esterified (24.0±10.7% hydrolysis, P<0.0001 for difference in hydrolysis between groups). This result demonstrates that CEL is the enzyme primarily responsible for hydrolysis of HDL-CE before delivery to the bile canaliculus.

### Example 3

### Biliary Lipid and Bile Salt Analyses

Cholesteryl ester and total cholesterol were measured directly using the fluorimetric method described by Mizoguchi et al. (Mizoguchi, et al., A method of direct measurement for the enzymatic determination of cholesteryl esters. J. Lipid Res. 45:396-401 (2004)) with some modifications. While cholesteryl ester was measured exactly as described, the method was modified to measure total cholesterol by leaving cholesterol oxidase out of the “FC (free cholesterol) decomposition reagent” so that both free and esterified cholesterol are measured in the second step. The fluorescence intensities were measured using a multi-well plate reader equipped with a filter for excitation and emission at 544 and 590 nm, respectively. Phospholipid concentration was determined by colorimetric assay (Wako, Richmond, Va.). Total bile acid concentration was determined by colorimetric assay (Trinity Biotech plc, Co. Wicklow, Ireland).

Since biliary cholesterol is derived from HDL free cholesterol and newly synthesized cholesterol in addition to HDL-CE, the effect of CEL on biliary cholesterol and cholesteryl ester mass was also determined. Gall bladder contents were collected from unfasted mice and the amount of cholesterol and cholesteryl ester was determined by fluorimetric enzyme assays. FIG. 2 shows that the mass of CE in gall bladder bile was ˜2 fold greater in Cel^{-/-} mice than in controls, both in absolute concentration (61.8±37.4 μM vs. 32.0±16.0 μM, P=0.023) and as a percent of total cholesterol (4.28±2.46% vs. 2.17±1.10%, P=0.016; n=13 and 8, respectively). Total biliary cholesterol concentration was not significantly different. Bile acid and phospholipid concentrations were also not significantly different between groups. Together, Examples 2 and 3 indicate that the absence of CEL causes significantly more CE from HDL to be transported directly into bile without hydrolysis.

### Example 4

### Greater Fecal Sterol Secretion by Cel^{-/-} Mice

Animals were individually housed in cages fitted with a wire platform with full access to food and water. After a 1 or 2 day acclimation period, feces were collected for 72 h and sterols were quantitated using a variation of the method described by Post et al. (Post, et al., Increased fecal bile acid excretion in transgenic mice with elevated expression of human phospholipid transfer protein. Arterioscler. Thromb. Vasc. Biol. 23:892-97 (2003)). After being dried and weighed, fecal material was ground to a fine powder. 5α-cholestane (40 μg) and [24-¹⁴C]-taurocholic acid (0.02 μCi) were added to 0.5 g of the ground fecal material as internal standards for extraction efficiency of neutral and acidic sterols, respectively. The fecal material plus standards were suspended in 10 volumes of alkaline methanol (0.2 M NaOH in 80% methanol), incubated at 80° C. for 2 hr, and neutral sterols were separated by 3 extractions with equal volumes of petroleum ether. The combined organic fractions were evaporated under nitrogen, resuspended in hexane, and a portion was used to measure neutral sterols by gas chromatography (Shimadzu Scientific Instruments). Data reported represent the sum of areas under the curves for cholesterol, coprostanol, and lathosterol. The aqueous residue from above was filtered, washed, dried, resuspended in water, and applied to prewashed C18 Bond Elut columns (Varian Inc., Palo Alto, Calif.). Bile acids were eluted from the columns with methanol, concentrated and quantitated by colorimetric assay (Trinity Biotech). To measure fecal sterol esters, ground fecal samples were resuspended in water, extracted with petroleum ether, concentrated, resolved by TLC as above and visualized at 80° C. after staining with 10% phosphomolybdic acid in ethanol, or bands were scraped and quantitated by scintillation spectrometry.

To determine if reverse cholesterol transport is increased to a physiological important degree in Cel^{-/-} mice, total excreted mass of both neutral and acidic (bile acids) sterols was quantitated for control and Cel^{-/-} mice. Data were normalized to output per day per gram of body weight (bw). As shown in FIG. 3A, fecal neutral sterol mass excreted by Cel^{-/-} mice was 59% greater than that excreted by control mice (131.2±25.6 nmole/d/gbw vs. 82.6±14.1 nmole/d/gbw, P<0.001, n=8 per group). Surprisingly, bile acid excretion by Cel^{-/-} mice was also 42% greater than that of control mice (76.8±23.6 nmole/d/gbw vs. 54.2±9.9 nmole/d/gbw for controls, P=0.034). Bile acid pool size did not differ between the two groups of mice (221±40 nmole/gbw for controls vs. 237±33 nmole/gbw for Cel^{-/-} mice) indicating that increased bile acid excretion represented an additional net loss of sterol from the body. To determine if the extra neutral sterol excreted by Cel^{-/-} mice was present as CE, neutral sterols were extracted from other portions of fecal material and analyzed by TLC. FIG. 3B shows that the mass of esterified cholesterol in feces was markedly greater in Cel^{-/-} mice.

### Example 5

### Greater Excretion of HDL-CE-Derived Cholesterol by Cel^{-/-} Mice

As a first step to determine if these changes in hepatic HDL-CE processing and bile composition affected reverse cholesterol transport, excretion of HDL-CE derived sterols was measured. Mice were injected with HDL-[³H]CE and total fecal output was collected for the subsequent 48 h. Neutral sterols were extracted and the amount of radiolabel quantitated. FIG. 4 (left panel) shows that Cel^{-/-} mice disposed of over 2 times more cholesterol derived from the radiolabeled HDL-CE than did controls (5242±1566 dpm, 1.63±0.26% of dose vs. 2234±568 dpm, 0.76±0.23% of dose; P<0.005). Since CEL is also made by the pancreas and is the only cholesterol esterase functional in the intestinal lumen, the fecal extracts were analyzed by TLC to determine if the unhydrolyzed HDL-CE in bile was being excreted intact. After resolution, the bands of free and esterified cholesterol were scraped from the TLC plate, and the radiolabel in each was quantitated. As shown in FIG. 4 (left panel), 44.6±7.9% of the radiolabeled fecal cholesterol from Cel^{-/-} mice remained esterified while only 9.9±2.4% was esterified in material from control mice. These data show that in the absence of CEL, more cholesteryl ester from HDL is transported directly through the liver to the bile and remains unhydrolyzed as it passes through the intestine. Since CE is very poorly absorbed without lumenal hydrolysis, Cel^{-/-} mice excrete more HDL-derived cholesterol than do controls, suggesting that reverse cholesterol transport is increased in the absence of CEL.

### Example 6

### Increased Reverse Cholesterol Transport from Macrophage in Cel^{-/-} Mice

Radiolabeled macrophage were prepared by a method similar to that described by Zhang et al. (Zhang, Y. Z., et al. Overexpression of apolipoprotein A-I promotes reverse cholesterol transport from macrophages to feces in vivo. Circulation 108:661-63 (2003)). Human LDL+IDL (1.006<ρ<1.063) was labeled with [³H]cholesteryl oleate (GE Life Sciences) by the CETP exchange method (Pittman, et al., A nonendocytic mechanism for the selective uptake of high density lipoprotein-associated cholesterol esters. J. Biol. Chem. 262:2443-50 (1987)), reisolated by isopycnic centrifugation, and then acetylated using acetic anhydride followed by concentration and extensive dialysis. The radiolabeled, acetylated lipoproteins were incubated with J774 cells in serum-free medium for 18 to 24 hr to achieve at least 0.5 dpm/cell. Cells were harvested, washed, resuspended in serum-free medium, and administered by intraperitoneal injection such that each mouse received ˜1×10⁶ cells and at least 0.5×10⁶ dpm. Tail blood was drawn at 24 and 48 hr to monitor plasma isotope levels, and total fecal output was collected every 24 hr for 3 days. Radiolabeled neutral sterols were extracted from feces as described above and quantitated by scintillation spectrometry. To measure radiolabel in acidic sterols, a portion of the aqueous residue was dried onto filter paper and oxidized in an OX700 Biological Oxidizer (R. J. Harvey Instrument Corporation, Tappan, N.Y.) with concomitant collection of the ³H in scintillation cocktail.

To more directly test for reverse cholesterol transport from peripheral tissues, including cells found in arterial plaque, the above procedure was applied to control and Cel^{-/-} mice. The data in FIG. 5A show that excretion of macrophage-derived cholesterol was greatly increased by the absence of CEL, with a combined increase of 37% over the 3 days of collection (375±56 dpm/gbw for controls vs. 512±85 dpm/gbw for Cel^{-/-} mice, P=0.001). FIG. 5B shows that excretion of bile acids derived from macrophage cholesterol was also increased 30% (1018±168 dpm/gbw for controls vs. 1326±170 dpm/gbw for Cel^{-/-} mice, P=0.003). These results demonstrate that whole body reverse cholesterol transport is elevated in Cel^{-/-} mice.

### Example 7

### Increased HDL in Cel^{-/-} Mice Expressing CETP

CETP transgenic mice (Marotti et al., Severe atherosclerosis in transgenic mice expressing simian cholesteryl ester transfer protein, Nature 364:73-75 (1993)) were purchased from Jackson Laboratories and crossed with Cel^{-/-} mice to generate a new strain carrying both genetic modifications in order to study the effects of CEL in animals with HDL metabolism that more closely models that of humans. Pooled plasma was prepared from chow-fed, unfasted control (n=12 and Cel^{-/-} mice (n=15) and fractionated by FPLC. The amount of cholesterol in each of the 75 fractions was determined by standard colorimetric assay. Data were analyzed to represent the amount of cholesterol in HDL, LDL, and TRL (triglyceride rich lipoproteins, VLDL+IDL) and expressed as a percent of total plasma cholesterol. As shown in FIG. 6, the relative amount of HDL was markedly increased while the relative amount of atherogenic TRL was dramatically deceased in Cel^{-/-} mice as compared to controls. The percent change from control values was +19.4% for HDL and −36.6% for TRL. Raising HDL and decreasing TRL to the extent seen in the CETP/Cel^{-/-} mice would substantially reduce risk for cardiovascular disease in a human clinical context.

### Example 8

### Increased Total and HDL-CE Excretion by Cel^{-/-} Mice Expressing CETP

Reverse cholesterol transport by CETP/Cel^{-/-} mice was determined by measuring mass of excreted neutral sterols and by measuring the excretion of radiolabel from HDL-[³H]CE using methods described in the Examples above. As illustrated in FIG. 7 (left panel) neutral sterol excretion was increased 67% in CETP/Cel^{-/-} mice relative to CETP/control mice (29.7+/−9.3 μg/d/gbw, n=14; vs. 17.8+/−4.6 μg/d/gbw, n=8; P=0.003). In keeping with this result, FIG. 7 (middle panel) shows that 24 hr excretion of neutral sterols from radiolabeled HDL-CE was similarly increased in CETP/Cel^{-/-} mice (142±47 dpm/gbw, n=13; vs. 83±38 dpm/gbw, n=8 CETP/controls; P=0.008). FIG. 7 (right panel) shows that, as with normal Cel^{-/-} mice, dramatically more of this radiolabel was esterified in the CETP/Cel^{-/-} mice as compared to the CETP/control mice (9.0±3.2%, n=15; vs. 1.7±1.4%, n=10; P<0.001). Thus, in the context of CETP expression lack of CEL in mice has the dual effect of raising HDL and also increasing reverse cholesterol transport. This result supports the concept that inhibitors of CEL would have beneficial effects in humans as well.

### Example 9

### CEL Inhibitors Increase Reverse Cholesterol Transport

The effect of (1,5-dimethylhexyl)carbamic acid 4-phenoxyphenyl ester on cholesterol disposal has been measured in C57BL/6 mice. Baseline daily fecal cholesterol disposal by chow-fed mice was measured over 3 days using established methods. Thereafter, the same mice were fed a chow diet to which had been added 1 milligram of (1,5-dimethylhexyl)carbamic acid 4-phenoxyphenyl ester per gram of food. The average body weight of the mice was 24 grams. Mice of this size are known to eat between 3.5 and 4 grams of chow per day. Thus, the amount of inhibitor consumed was between 3.5 and 4.0 milligrams per mouse per day or between 146 and 167 milligrams per kilogram of bodyweight. After eating inhibitor-containing diet for 2 weeks, daily fecal cholesterol excretion was again measured over 3 days. As illustrated in FIG. 8, paired data analysis revealed that cholesterol excretion was significantly increased by (1,5-dimethylhexyl)carbamic acid 4-phenoxyphenyl ester treatment (P=0.01). The average excretion at baseline was 48.9±11.3 micrograms per day per gram bodyweight (range 28.6 to 71.8), which increased to 62.1±10.4 micrograms per day per gram bodyweight (range 45.3 to 89.1) after treatment. The average change per mouse was +33.0% (range −19.3 to +95.4), with 10 of the 13 mice responding positively, 1 showing no change and 2 responding negatively). Of those that responded positively to treatment, the average increase of was 46.7% over baseline. These results demonstrate that (1,5-dimethylhexyl)carbamic acid 4-phenoxyphenyl ester recapitulates at least one of the beneficial phenotypes observed in Cel^{-/-} mice—an increase in reverse cholesterol transport, as measured by increased excretion of cholesterol from the body.

### Example 10

### CEL siRNA Effect on Reverse Cholesterol Transport

Wild type C57BL/6 mice fed a standard chow diet are divided into two groups: recombinant lentivirus containing CEL siRNA is injected into the test group and vehicle only is injected into the control group. After 48 hr, all mice receive an intraperitoneal injection of murine macrophage cells that have been loaded with radiolabeled cholesteryl ester from acetylated LDL. Neutral and acidic sterols are extracted from total feces collected for 3 days subsequent to the injection, and the amount of radiolabel in each fraction is determined by scintillation spectrometry. Results show more radioactive sterol in both the neutral and acidic fractions of material collected from mice treated with the siRNA than in material collected from mice in the control group.

### Example 11

### Therapeutic Use of a CEL Inhibitor

A patient with suboptimal HDL cholesterol level, which is not the result of a known genetic or other predisposing condition, is prescribed (1,5-dimethylhexyl)carbamic acid 4-phenoxyphenyl ester to raise serum HDL levels. The patient takes a daily oral dose of 1 mg per kg body of weight for a period of two weeks, after which serum cholesterol in HDL, LDL, and VLDL is again measured. It is determined that HDL cholesterol has increased by 15-20%, that the ratio of HDL:LDL has increased, and that VLDL is decreased, such that risk for cardiovascular disease is reduced.

### Example 12

### Therapeutic Use of a CEL Inhibitor

A patient with suboptimal HDL cholesterol level, which is not the result of a known genetic or other predisposing condition, is prescribed 4-methyl-1-piperidinecarboxylic acid 4-phenoxyphenyl ester to raise serum HDL levels. The patient takes a daily oral dose of 1 mg per kg body of weight for a period of two weeks, after which serum cholesterol in HDL, LDL, and VLDL is again measured. It is determined that HDL cholesterol has increased by 15-20%, that the ratio of HDL:LDL has increased, and that VLDL is decreased, such that risk for cardiovascular disease is reduced.

All documents cited are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of raising serum HDL levels comprising administering to a subject in need thereof a safe and effective amount of at least one carboxyl ester lipase (CEL) inhibitor.

2. The method of claim 1 wherein the at least one CEL inhibitor is selected from the group consisting of (1,5-dimethylhexyl)carbamic acid 4-phenoxyphenyl ester and 4-methyl-1-piperidinecarboxylic acid 4-phenoxyphenyl ester.

3. The method of claim 2 wherein a second active ingredient is co-administered to the subject, wherein the second active ingredient is a cholesterol lowering agent.

4. The method of claim 3 wherein the second active ingredient is a statin drug.

5. The method of claim 4 wherein the statin drug is selected from the group consisting of atorvastatin, cerivastatin, fluvastatin, lovastatin, mevastatin, pitavastatin, pravastatin, rosuvastatin, and simvastatin.

6. A pharmaceutical composition comprising:
(a) at least one carboxyl ester lipase (CEL) inhibitor; and
(b) at least one pharmaceutically-acceptable carrier,
wherein said pharmaceutical composition acts to raise serum HDL levels in a subject in need thereof.

7. The pharmaceutical composition of claim 6 wherein the at least one CEL inhibitor is selected from the group consisting of (1,5-dimethylhexyl)carbamic acid 4-phenoxyphenyl ester and 4-methyl-1-piperidinecarboxylic acid 4-phenoxyphenyl ester.

8. The pharmaceutical composition of claim 7 further comprising a second active ingredient, wherein the second active ingredient is a cholesterol lowering agent.

9. The pharmaceutical composition of claim 8 wherein the second active ingredient is a statin drug.

10. The pharmaceutical composition of claim 9 wherein the statin drug is selected from the group consisting of atorvastatin, cerivastatin, fluvastatin, lovastatin, mevastatin, pitavastatin, pravastatin, rosuvastatin, and simvastatin.

11. A method of increasing reverse cholesterol transport comprising administering to a subject in need thereof a safe and effective amount of at least one carboxyl ester lipase (CEL) inhibitor.

12. The method of claim 11 wherein the at least one CEL inhibitor is selected from the group consisting of (1,5-dimethylhexyl)carbamic acid 4-phenoxyphenyl ester and 4-methyl-1-piperidinecarboxylic acid 4-phenoxyphenyl ester.

13. The method of claim 12 wherein a second active ingredient is co-administered to the subject, wherein the second active ingredient is a cholesterol lowering agent.

14. The method of claim 13 wherein the second active ingredient is a statin drug.

15. The method of claim 14 wherein the statin drug is selected from the group consisting of atorvastatin, cerivastatin, fluvastatin, lovastatin, mevastatin, pitavastatin, pravastatin, rosuvastatin, and simvastatin.

16. A pharmaceutical composition comprising:
(a) at least one carboxyl ester lipase (CEL) inhibitor; and
(b) at least one pharmaceutically-acceptable carrier,
wherein said pharmaceutical composition acts to increase reverse cholesterol transport in a subject in need thereof.

17. The pharmaceutical composition of claim 16 wherein the at least one CEL inhibitor is selected from the group consisting of (1,5-dimethylhexyl)carbamic acid 4-phenoxyphenyl ester and 4-methyl-1-piperidinecarboxylic acid 4-phenoxyphenyl ester.

18. The pharmaceutical composition of claim 17 further comprising a second active ingredient, wherein the second active ingredient is a cholesterol lowering agent.

19. The pharmaceutical composition of claim 18 wherein the second active ingredient is a statin drug.

20. The pharmaceutical composition of claim 19 wherein the statin drug is selected from the group consisting of atorvastatin, cerivastatin, fluvastatin, lovastatin, mevastatin, pitavastatin, pravastatin, rosuvastatin, and simvastatin.

21. A method of increasing reverse cholesterol transport comprising administering to a subject in need thereof a safe and effective amount of a CEL inhibitor selected from the group consisting of (1,5-dimethylhexyl)carbamic acid 4-phenoxyphenyl ester and 4-methyl-1-piperidinecarboxylic acid 4-phenoxyphenyl ester.

22. The method of claim 21, wherein a statin drug is co-administered to the subject.

23. The method of claim 22, wherein the statin drug is selected from the group consisting of atorvastatin, cerivastatin, fluvastatin, lovastatin, mevastatin, pitavastatin, pravastatin, rosuvastatin, and simvastatin.

24. A method for treating disease by increasing reverse cholesterol transport, wherein the disease is selected from the group consisting of atherosclerosis, hyperlipidemia, hypercholesterolemia, cholestatic liver disease, peripheral vascular disease, dyslipidemia, hyperbetalipoproteinemia, hypoalphalipoproteinemia, hypertriglyceridemia, familial-hypercholesterolemia, cardiovascular disorders, angina, ischemia, cardiac schemia, stroke, myocardial infarction, reperfusion injury, angioplastic restenosis, hypertension, vascular complications of diabetes, obesity and endotoxemia, which comprises administering a safe and effective amount of a carboxyl ester lipase (CEL) inhibitor to a subject in need thereof.

25. The method of claim 24 wherein the CEL inhibitor is selected from the group consisting of (1,5-dimethylhexyl)carbamic acid 4-phenoxyphenyl ester and 4-methyl-1-piperidinecarboxylic acid 4-phenoxyphenyl ester.

26. The method of claim 25 wherein a statin drug is co-administered to the subject.

27. A method for treating disease by raising serum HDL levels, wherein the disease is selected from the group consisting of atherosclerosis, hyperlipidemia, hypercholesterolemia, cholestatic liver disease, peripheral vascular disease, dyslipidemia, hyperbetalipoproteinemia, hypoalphalipoproteinemia, hypertriglyceridemia, familial-hypercholesterolemia, cardiovascular disorders, angina, ischemia, cardiac schemia, stroke, myocardial infarction, reperfusion injury, angioplastic restenosis, hypertension, vascular complications of diabetes, obesity and endotoxemia, which comprises administering a safe and effective amount of a carboxyl ester lipase (CEL) inhibitor to a subject in need thereof.

28. The method of claim 27 wherein the CEL inhibitor is selected from the group consisting of (1,5-dimethylhexyl)carbamic acid 4-phenoxyphenyl ester and 4-methyl-1-piperidinecarboxylic acid 4-phenoxyphenyl ester.

29. The method of claim 28 wherein a statin drug is co-administered to the subject.
